(19) United States
(12) Patent Application Publication
(10) Pub. No.: US 2020/0046570 A1
(43) Pub. Date: Feb. 13, 2020

(54) PATIENT MEDICAL ACCESS OPENING PROTECTION DEVICE

(71) Applicant: MKZ Holdings, LLC, Frankenmuth, MI (US)

(72) Inventors: Bruce Sheridan, Cass City, MI (US); Susanne Sheridan, Cass City, MI (US)

(21) Appl. No.: 16/492,681

(22) PCT Filed: Mar. 14, 2018
(86) PCT No.: PCT/US18/22427

§ 371 Date: Sep. 10, 2019

Related U.S. Application Data
(60) Provisional application No. 62/470,928, filed on Mar. 14, 2017.

(51) Int. Cl. A61F 13/02; A61L 15/58; A61M 25/02

(52) U.S. Cl. CPC A61F 13/0243; A61F 13/0233; A61F 2013/00268; A61L 15/58; A61M 25/02; A61F 13/0236

(57) ABSTRACT

A medical access opening protection device including an annular adhesive band carried on an outer perimeter region of a moisture-impervious film, a release liner carried by the adhesive band, and an annular barrier band of barrier material disposed concentrically within the adhesive band. The moisture impervious film may have sufficiently high maximum elongation value and ultimate tensile strength to accommodate medical equipment without breaking and without causing the adhesive band to break contact with a user's skin. Alternatively, or in addition, the film may comprise an annular flange and blister surrounded by the annular flange, and the annular adhesive band is disposed on the annular flange.

### CROSS-REFERENCES TO RELATED APPLICATIONS

This application claims priority from Patent Cooperation Treaty Patent Application serial number PCT/US18/22427 filed on Mar. 14, 2018 and U.S. Provisional Patent Application Ser. No. 62/470,028 filed on Mar. 14, 2017.

### STATEMENT REGARDING FEDERALLY SPONSORED RESEARCH OR DEVELOPMENT

Not Applicable

### BACKGROUND

This application relates generally to a device for protecting a medical access opening in a patient.

### DESCRIPTION OF RELATED ART INCLUDING INFORMATION DISCLOSED UNDER 37 CFR 1.97 AND 1.98

Patients who have medical access openings in their bodies and find it necessary or advisable to expose themselves to water, soap, and other cleansing products for the purpose of bathing, are known to protect their medical access openings and associated medical equipment against intrusion by moisture and other contaminants by securing waterproof material over the access openings. Patients are known to either apply a shower barrier product such as HydroSeal (available from 2GMedical, LLC) for this purpose, or to simply tape plastic over their medical access openings.

### SUMMARY

A medical access opening protection device is provided for protecting a patient's medical access opening or surgical site and associated medical equipment from moisture in a wet environment. The device may include a moisture-impervious film, an annular adhesive band carried by the film and disposed on an outer perimeter region of the film, a release liner carried by the adhesive band, and an annular barrier band of barrier material disposed concentrically within the adhesive band. The moisture impervious film may have sufficiently high maximum elongation value and ultimate tensile strength to accommodate medical equipment without breaking and without causing the adhesive band to break contact with a user's skin.

Also provided is a medical access opening protection device for shielding a patient's medical access opening or surgical site and associated medical equipment from moisture in a wet environment, in which the device comprises a moisture-impervious film comprising an annular flange and blister surrounded by the annular flange, an annular adhesive band carried by the film and disposed on the annular flange of the film, a release liner carried by the adhesive band, and an annular barrier band of barrier material disposed concentrically within the adhesive band.

Also provided is a method for shielding a patient's medical access opening or surgical site and associated medical equipment from moisture. The method includes providing a cover comprising a moisture impervious film, an annular adhesive band of material carried by the film and disposed on an outer perimeter region of the film, a release liner carried by the adhesive band, and a barrier band of barrier material placed concentrically within the adhesive band; placing the cover over the access opening such that the release liner is seated against a user's skin; and peeling the release liner back to expose and adhere the adhesive band to the user's skin by keeping the cover pressed against the user's skin as the release liner is being removed.

### DRAWING DESCRIPTIONS

These and other features and advantages will become apparent to those skilled in the art in connection with the following detailed description and drawings of one or more embodiments of the invention, in which:

FIG. 1 is a front view of a first illustrative embodiment of a medical access opening protection device;

FIG. 2 is a cross-sectional edge view of the medical access opening protection device of FIG. 1, taken along line 2-2 of FIG. 1;

FIG. 3 is a front view of an alternative square configuration of the first illustrative embodiment;

FIG. 4 is a front view of a second illustrative embodiment of a medical access opening protection device;

FIG. 5 is a cross-sectional edge view of the medical access opening protection device of FIG. 4, taken along line 5-5 of FIG. 4;

FIG. 6 is a front view of an alternative square configuration of the second illustrative embodiment;

FIG. 7 is a front view of a third illustrative embodiment of a medical access opening protection device;

FIG. 8 is a cross-sectional edge view of the medical access opening protection device of FIG. 7, taken along line 8-8 of FIG. 7;

FIG. 9 is a front view of an alternative square configuration of the third illustrative embodiment; and

FIG. 10 is a flow chart showing a method for applying a medical access opening protection device.

### DETAILED DESCRIPTION

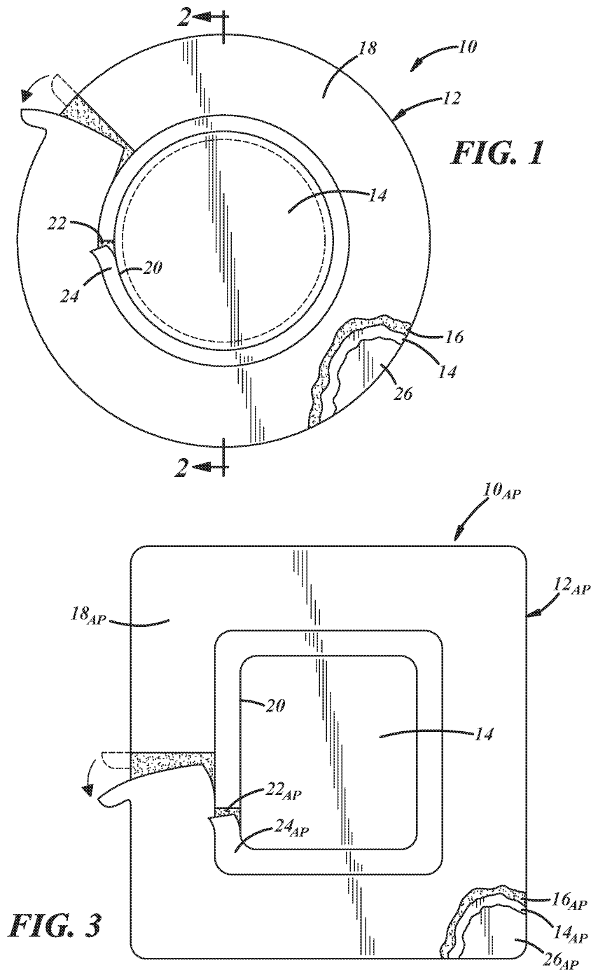

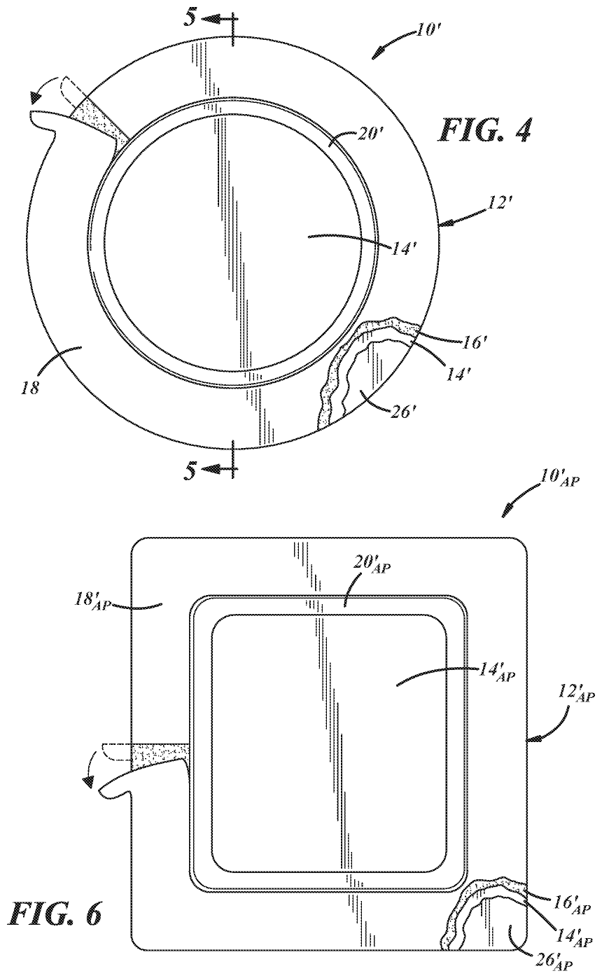

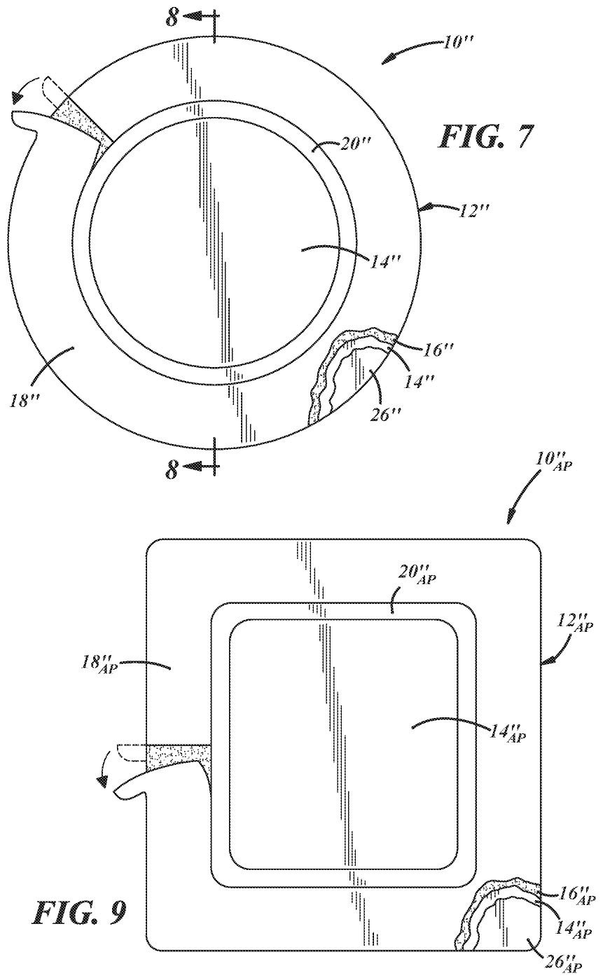

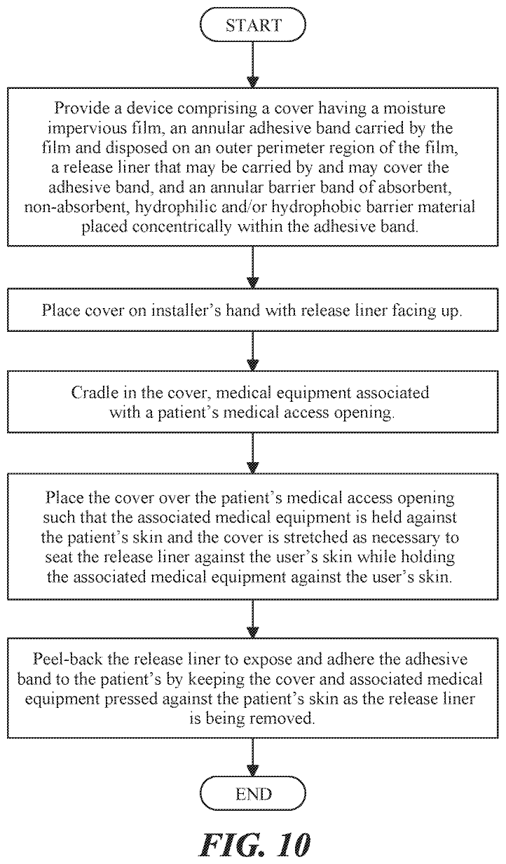

A first embodiment of a device for protecting a medical access opening or surgical site in a patient, and also associated medical equipment such as a central venous access device (PORT) or a peripherally inserted central catheter (PICC) line; from moisture in a wet environment such as a shower, bath, sponge bathing, rain, swimming or athletic activities; is generally shown at 10 in FIGS. 1-3 of the appended drawings. A second embodiment of a patient access opening protection device is generally shown at 10′ in FIGS. 4-6 of the drawings. A third embodiment of a patient access opening protection device is generally shown at 10″ in FIGS. 7-9 of the drawings. Reference numerals with the designation prime (′) in FIGS. 1-3 and with the designation double-prime (″) in FIGS. 4-6 indicate alternative configurations of elements that also appear in the first embodiment. Unless indicated otherwise, where a portion of the following description uses a reference numeral to refer to elements of the first embodiment in FIGS. 1-3, that portion of the description applies equally to elements of the second embodiment designated by primed numerals in FIGS. 3-6 and elements of the third embodiment designated by double-primed numerals in FIGS. 7-9. Alternate planforms for each of the three embodiments are shown in FIGS. 3, 6, and 9, respectively, in which reference numeral include an “AP” (an) subscript.

The device 10 may include a cover 12 comprising moisture impervious film 14. The moisture impervious film 14 may have sufficiently high maximum elongation value and ultimate tensile strength to accommodate, i.e., stretch around and hold and retain, medical dressings and equipment such as tubing, without breaking and without causing the adhesive band to break contact with a user's skin. The moisture impervious film 14 may be a breathable urethane film such as Product No. 8166 urethane film available from Medco Coated Products of Bedford Heights, Ohio. The moisture impervious film 14 may preferably have a maximum elongation value of 400-500%. The film 14 may also or alternatively have an ultimate tensile strength of 4200-5200 psi. The film 14 may be formed via, for example, vac-forming, to include a receptacle or pouch for receiving and retaining dressings, tubing, and any other gear associated with a patient's medical access opening. The film 14 may be sized large enough for the receptacle to cover a 4″×4¾″ Tegaderm® patch, i.e., approximately 12.75″ in diameter, (but may be smaller or larger to accommodate a variety of patient anatomies, pediatric patients or obese patients).

An annular adhesive band 16 may be carried by the film 14 and positioned on an outer perimeter region of the film 14. The adhesive band 16 may comprise a pressure-sensitive adhesive and may be adhered to the film 14 via the adhesive band's own adhesive properties or by any other suitable means. A release liner 18 is carried by and may cover the adhesive band 16 to protect the adhesive band 16 from contaminants and may be easily removable to expose the adhesive band 16 for application to a patient. The release liner 18 may comprise an annular sheet of paper any suitable flexible material and may be shaped to include pull tabs that extend radially outward beyond a perimeter edge of the adhesive band 16 to allow a user to more easily grasp and remove the release liner 18.

The device 10 may include adhesive transfer tape, which may comprise both the annular adhesive band 16 and release liner 18. The adhesive transfer tape may be of any suitable type including, for example, product no. 8251 transfer tape available from Medco Coated Products. The adhesive transfer tape may be 2.0-mil in thickness and may include a medical acrylic pressure-sensitive adhesive film 14 carrying the release liner 18. The adhesive transfer tape may preferably have a loop tack strength of approximately 5.98 lbs., per the PSTC-16 standard established by the Pressure Sensitive Tape Council.

A barrier band 20 comprising an annular band of barrier material such as absorbent and/or hydrophilic material may be carried by the film 14 and disposed concentrically within the adhesive band 16. The barrier band 20 may comprise absorbent and/or hydrophilic material such that, when the cover 12 is placed over a medical access opening on a user's body, the absorbent and/or hydrophilic barrier material of the barrier band 20 will absorb and/or attract any moisture that might leak past or through the adhesive band 16. The barrier band 20 may be adhered to the film 14 by an adhesive, by vac-forming the film 14 around one or more surfaces of the barrier band 20, and/or by any other suitable means. The absorbent or hydrophilic barrier material may comprise any suitable foam or sponge material such as, for example, cellulose sponge material such as that available from, for example, 3M Company of St. Paul, Minn. The barrier band 20 may also include an adhesive layer 22 positioned for adhesion to a patient's skin, and a release liner 24 to prevent contamination of the adhesive layer 22 prior to application. While the barrier band adhesive layer 22 and release liner 24 are shown only in the embodiment of FIGS. 1-3, other embodiments, including the embodiments of FIGS. 4-6 and 7-9 may include these features as well.

Alternatively, or in addition, the barrier band 20 may comprise non-absorbent or hydrophobic material such that, when the cover 12 is placed over a medical access opening on a user's body, the non-absorbent or hydrophobic barrier material will resist and/or repel any moisture that might leak past of through the adhesive band 16. The non-absorbent or hydrophobic barrier material may comprise any suitable non-absorbent foam, and/or any suitable hydrophobic material such as, for example, a general purpose cross-linked polyethylene foam (XLPE) such as 2A Volara available from the Atlantic Gasket Corporation.

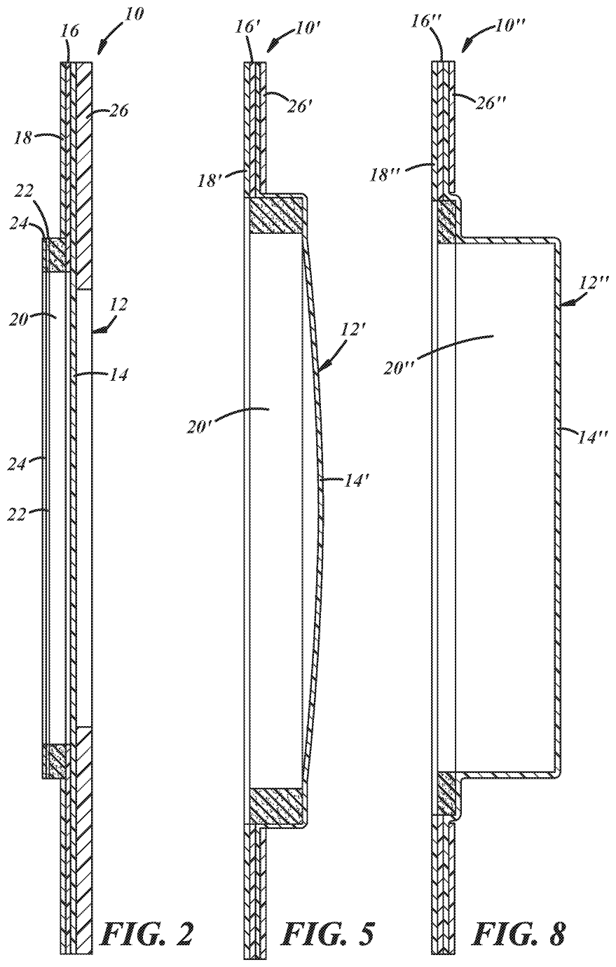

As is also shown in FIGS. 1 and 2, the device 10 may include a flange support ring 26, which may comprise an annular sheet of urethane or any suitable flexible material and a low to mid-range-tack adhesive that adheres the flange support ring 26 to an outer surface of the flange. The flange support ring 26 helps to maintain the shape of the flange to ease handling and application of the device 10 to a patient.

As shown in FIG. 3, the device 10_{AP} including its film 14_{AP}, adhesive band 16_{AP}, barrier band 20_{AP}, release liner 18_{AP}, and flange support ring 26_{AP}; may all have square planforms. In other embodiments, these elements may have any suitably-shaped planform.

According to the second embodiment, and as shown in FIGS. 4 and 5, the moisture-impervious film 14′ of the device 10′ may comprise an annular flange and blister surrounded by the annular flange, with the annular adhesive band 16′ being carried by the film 14′ in a position disposed on the annular flange of the film 14′. The blister may be vac-formed into the film 14′ or may be formed by any other suitable means. Further according to this embodiment, the barrier band 20′ of the device 10′ may be disposed within the blister and adjacent an inner peripheral wall of the blister. The barrier band 20′ may fixed to an inner surface of the blister by, for example, a layer of adhesive material and/or by vac-forming the film 14′ over and around the barrier band 20′.

Further according to the second embodiment, and as shown in FIG. 6, the cover 12′_{AP} of the device 10′_{AP}, including its film 14′_{AP}, adhesive band 16′_{AP}, barrier band 20′_{AP}, release liner 18′AP, and flange support ring 26′_{AP}; may all have square, rather than circular, planforms. In other embodiments, these elements may have any suitably-shaped planform.

According to the third embodiment, and as shown in FIGS. 7 and 8, the blister of the device 10″ may include an annular mezzanine of lesser depth than a central portion of the blister, and the barrier band 20″ may be disposed on the annular mezzanine. The mezzanine may be vac-formed into the film 14″ along with the rest of the blister or may be formed by any other suitable means. The barrier band 20″ of the device 10″ may fixed to the mezzanine by, for example, a layer of adhesive material and/or by vac-forming the film 14″ over and around the barrier band 20″.

Further according to the third embodiment, and as shown in FIG. 9, the cover 12″_{AP} of the device 10″_{AP}, including its film 14″_{AP}, adhesive band 16″_{AP}, barrier band 20″_{AP}, release liner 18″_{AP}, and flange support ring 26″_{AP}; may all have square, rather than circular planforms. In other embodiments, these elements may have any suitably-shaped planform.

In practice, a patient's access opening and associated medical equipment can be shielded from moisture in a wet environment such as a shower, by first providing a device 10 comprising a cover 12, such as the ones described above, that comprises a moisture impervious film 14, an annular adhesive band 16 carried by the film 14 and disposed on an outer perimeter region of the film 14, a release liner 18 that may be carried by and may cover the adhesive band 16, and an annular barrier band 20 of absorbent, non-absorbent, hydrophilic and/or hydrophobic barrier material placed concentrically within the adhesive band 16. Associated medical equipment such as dressings and tubing associated with PORT and PICC devices may then be cradled in the cover 12 in a user's hand, and the cover 12 placed over the user's access opening such that the associated medical equipment is held against the user's skin and the cover 12 is stretched as necessary to seat the release liner 18 against the user's skin while holding the associated medical equipment against the user's skin. The release liner 18 may then be peeled back to expose and adhere the adhesive band 16 to the user's skin by keeping the cover 12 and associated medical equipment pressed against the user's skin as the release liner 18 is being removed.

A device constructed and used as disclosed above, in addition to protecting a medical access opening or surgical site from moisture, reduces or eliminates discomfort associated with pressure on the medical access/surgical site and associated medical equipment (such as a central line, PICC or port, etc.).

This description, rather than describing limitations of an invention, only illustrates embodiments of the invention recited in the claims. The language of this description is therefore exclusively descriptive and is non-limiting.

Obviously, it's possible to modify this invention from what the description teaches. Within the scope of the claims, one may practice the invention other than as described above.

What is claimed is:

1. A medical access opening protection device for protecting a patient's medical access opening or surgical site and associated medical equipment from moisture in a wet environment, the device comprising:
a moisture-impervious film;
an annular adhesive band carried by the film and disposed on an outer perimeter region of the film;
a release liner carried by the adhesive band;
an annular barrier band of barrier material disposed concentrically within the adhesive band; and
the moisture impervious film having sufficiently high maximum elongation value and ultimate tensile strength to accommodate medical equipment without breaking and without causing the adhesive band to break contact with a user's skin.

2. A medical access opening protection device as defined in claim 1 in which the moisture impervious film is a breathable urethane film.

3. A medical access opening protection device as defined in claim 1 further comprising adhesive transfer tape comprising the annular adhesive band and release liner.

4. A medical access opening protection device as defined in claim 3 in which the adhesive transfer tape is a medical acrylic pressure-sensitive adhesive film carrying release liner.

5. A medical access opening protection device as defined in claim 3 in which the adhesive transfer tape has a loop tack strength of 5.98 lbs.

6. A medical access opening protection device as defined in claim 1 in which the barrier band comprises cellulose sponge.

7. A medical access opening protection device as defined in claim 1 in which the moisture impervious film has a maximum elongation value of 400-500%.

8. A medical access opening protection device as defined in claim 1 in which the moisture impervious film has an ultimate tensile strength of 4200-5200 psi.

9. A medical access opening protection device as defined in claim 1 in which:
the moisture-impervious film comprises an annular flange and a blister surrounded by the annular flange; and
the annular adhesive band is carried by the film and is disposed on the annular flange of the film.

10. A medical access opening protection device for shielding a patient's medical access opening or surgical site and associated medical equipment from moisture in a wet environment, the device comprising:
a moisture-impervious film comprising an annular flange and blister surrounded by the annular flange;
an annular adhesive band carried by the film and disposed on the annular flange of the film;
a release liner carried by the adhesive band; and
an annular barrier band of barrier material disposed concentrically within the adhesive band.

11. A medical access opening protection device as defined in claim 10, in which the moisture impervious film has sufficiently high maximum elongation value and ultimate tensile strength to accommodate medical equipment without breaking and without causing the adhesive band to break contact with a user's skin.

12. A medical access opening protection device as defined in claim 10 in which the band of barrier material is disposed within the blister.

13. A medical access opening protection device as defined in claim 12 in which:
the blister includes an annular mezzanine (of lesser depth than a central portion of the blister); and
the barrier band is disposed on the annular mezzanine.

14. A medical access opening protection device as defined in claim 13 in which the barrier band is fixed to the mezzanine.

15. A method for shielding a patient's medical access opening or surgical site and associated medical equipment from moisture, the method including the steps of:
providing a cover comprising a moisture impervious film, an annular adhesive band of material carried by the film and disposed on an outer perimeter region of the film, a release liner carried by the adhesive band, and a barrier band of barrier material placed concentrically within the adhesive band;
placing the cover over the access opening such that the release liner is seated against a user's skin; and
peeling the release liner back to expose and adhere the adhesive band to the user's skin by keeping the cover pressed against the user's skin as the release liner is being removed.
